(19) United States
(12) Patent Application Publication
(10) Pub. No.: US 2015/0190244 A1
(43) Pub. Date: Jul. 9, 2015

(54) Breast Prosthesis with Pocket

(71) Applicant: Robert Halley, Atlanta, GA (US)

(72) Inventor: Robert Halley, Atlanta, GA (US)

(73) Assignee: AMERICAN BREAST CARE, LP, Marietta, GA (US)

(21) Appl. No.: 14/584,140

(22) Filed: Dec. 29, 2014

Related U.S. Application Data
(60) Provisional application No. 61/924,935, filed on Jan. 8, 2014.

(51) Int. Cl. A61F 2/52; B29C 45/00; A61F 2/50

(52) U.S. Cl. CPC A61F 2/52; A61F 2/5044; B29C 45/0003; B29C 45/0055; A61F 2002/523; B29C 2045/0058; B29K 2019/00

(57) ABSTRACT

A breast prosthesis includes a first layer with two films that are sealed together peripherally that define a front chamber. A second layer includes two films that are sealed together peripherally that define a back chamber. The second layer is adjacent to the first layer and defines a pocket therebetween. A portion of a peripheral edge of the second layer is sealed to a portion of a peripheral edge of the first layer to define a first opening so that the pocket defined between the first layer and the second layer opens to the first opening. The first opening and the pocket allow the user to stuff a material into the pocket through the first opening. A first silicone rubber is disposed in the front chamber and is cured in an outer shape of a breast. A second silicone rubber is disposed in the back chamber.

### CROSS-REFERENCE TO RELATED APPLICATION(S)

This application claims the benefit of U.S. Provisional Patent Application Ser. No. 61/924,935, filed Jan. 8, 2014, the entirety of which is hereby incorporated herein by reference.

### BACKGROUND OF THE INVENTION

1. Field of the Invention

The present invention relates to breast prostheses and, more specifically, to a breast prosthesis that includes a pocket into which material may be optionally placed.

2. Description of the Related Art

Many women who have had a mastectomy wear a breast prosthesis at the affected site. Typically, a breast prosthesis is made to look like a natural breast. It is placed against the patient's chest and is typically supported by a brassiere.

A common type of breast prosthesis is made from a soft silicone gel. Such a prosthesis includes an envelope made from a thin plastic film into which uncured silicone gel is injected. The pouch and the uncured silicone gel are then placed in a metallic mold having an interior that is complementary in shape to the desired shape of the prosthesis. The mould is then placed in an oven, where the silicone gel is heated until it is cured.

The silicone gel must be cured until it has a firmness so that it will maintain the shape of a natural breast during regular use. However, some users desire to adjust the character of the prosthesis, such as its shape, but are not able to do so.

Therefore, there is a need for a breast prosthesis that can be adjusted by the user.

### SUMMARY OF THE INVENTION

The disadvantages of the prior art are overcome by the present invention which, in one aspect, is a breast prosthesis for external wearing by a user that includes a first layer that includes two films that are sealed together peripherally so as to define a front chamber. A second layer includes two films that are sealed together peripherally so as to define a back chamber. The second layer is disposed adjacent to the first layer so that they define a pocket therebetween. A portion of a peripheral edge of the second layer is sealed to a corresponding portion of a peripheral edge of the first layer so as to define a first opening so that the pocket defined between the first layer and the second layer opens to the first opening. The first opening and the pocket are configured to allow the user to stuff a material into the pocket through the first opening. A first silicone rubber is disposed in the front chamber and is cured in an outer shape of a breast. A second silicone rubber is disposed in the back chamber.

In another aspect, the invention is a method of making a breast prosthesis, in which a first film sheet, a second film sheet, a third film sheet and a fourth film sheet are placed together. A non-stick shape is placed between the second film and the third film. The non-stick shape includes at least one protrusion extending therefrom. The first film sheet, the second film sheet, the third film sheet and the fourth film sheet are welded along a peripheral weld that is made discontinuous by the at least one protrusion extending from the non-stick shape so as to form an envelope in which the first film sheet and the second film sheet define a first chamber therebetween that opens to a first sprue. Similarly, the third film sheet and the fourth film sheet define a second chamber therebetween that opens to a second sprue. The second film sheet and the third film sheet define a pocket therebetween that opens to at least one first opening resulting from the weld being made discontinuous by the at least one protrusion. The envelope is placed in a breast form mold having a cavity that is complementary in shape to the breast prosthesis. A first silicone gel into the first chamber through the first sprue. A second silicone gel is injected into the second chamber through the second sprue. The first silicone gel is cured so as to form a first silicone rubber and the second silicone gel is cured so as to form a second silicone rubber, thereby forming the breast prosthesis. The breast prosthesis is removed from the mold. The non-stick shape is removed from the pocket.

In yet another aspect, the first film sheet and a second film sheet are welded together along a first weld to form a first sub-envelope in which the first film sheet and the second film sheet define a first chamber therebetween that opens to a first sprue. Similarly, the third film sheet and the fourth film sheet are welded together along a second weld so as to form a second sub-envelope in which the third film sheet and the fourth film sheet define a second chamber therebetween that opens to a second sprue. The first sub-envelope and the second sub-envelope are placed together so that the first weld is aligned with the second weld and the non-stick shape place between the first envelope and the second envelope. The first sub-envelope and the second sub-envelope are welded along a peripheral weld so as to generate the envelope. The peripheral weld is made discontinuous by the at least one protrusion extending from the non-stick shape so as to define a pocket therebetween that opens to at least one first opening resulting from the peripheral weld being made discontinuous by the at least one protrusion. The envelope is placed in a breast form mold having a cavity that is complementary in shape to the breast prosthesis. The first silicone gel is injected into the first chamber through the first sprue and the second silicone gel is injected into the second chamber through the second sprue. The first silicone gel is cured so as to form a first silicone rubber and the second silicone gel is cured so as to form a second silicone rubber, thereby forming the breast prosthesis. The breast prosthesis is removed from the mold. The non-stick shape is removed from the pocket.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1A is a front elevational view of one embodiment of a breast prosthesis with a pocket.

FIG. 1B is a side elevational view of the breast prosthesis shown in FIG. 1A.

FIG. 2 is a cross-sectional view of the breast prosthesis shown in FIG. 1 taken along line 2-2.

FIG. 3 is a front elevational view of a second embodiment of a breast prosthesis including three openings to the pocket.

FIGS. 4A-4G are a series of schematic drawings showing one embodiment method of making a breast prosthesis.

FIGS. 5A-5B are flowcharts showing two embodiments of a method of making a breast form.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

U.S. Pat. Nos. 4,247,351 and 4,249,975 disclose prostheses of silicone gel encased in polyurethane film and, therefore, are incorporated herein by reference. U.S. Pat. Nos. 4,950,291, 5,895,423, 5,922,023 and 7,759,354 disclose multi-chambered breast prosthesis and methods of making the same and are also, therefore, incorporated herein by reference.

One embodiment of a breast prosthesis includes two layers of silicone gel enclosed in polyurethane film. The shape of the prosthesis conforms to the shape of a female breast. The layers of silicone are arranged such that a layer of soft conformable silicone is closest to the chest of the wearer and can conform to any irregularities in the chest. The outer layer of silicone positioned in the front of prosthesis (furthest away from the body) is firm enough to give the prosthesis a natural look and a symmetric appearance. A pocket is formed between the inner layer and the outer layer to allow the user to adjust the character of the prosthesis by placing additional material of the user's choosing in the pocket

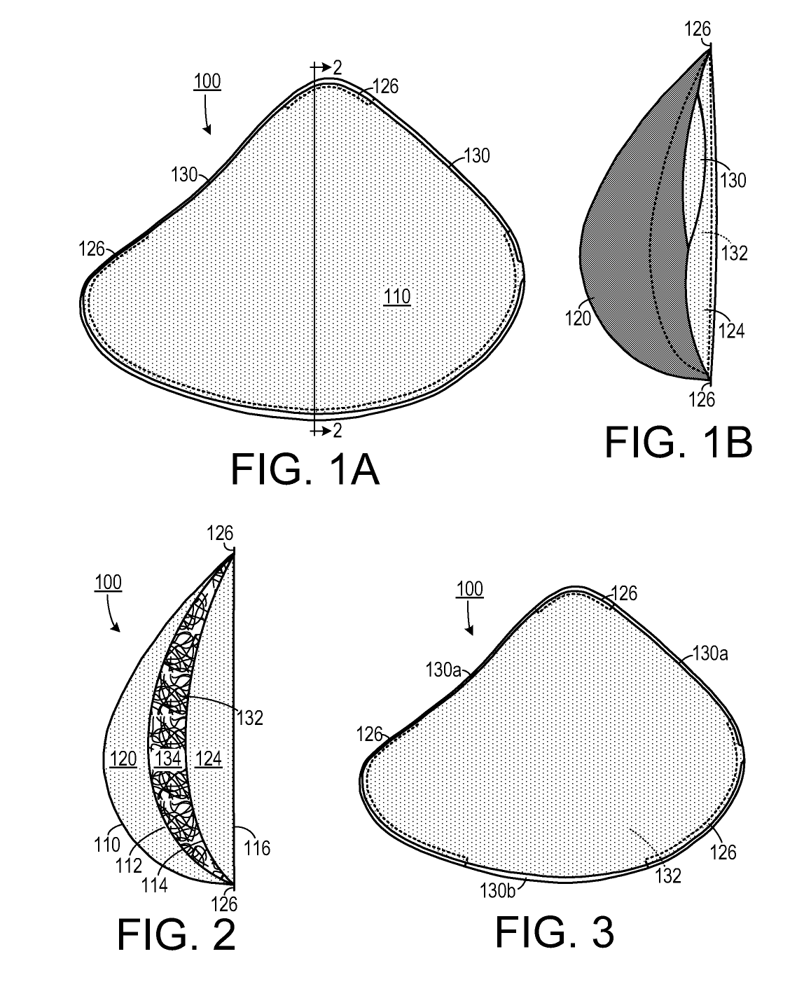

As shown in FIGS. 1A, 1B and 2, one embodiment of a layer breast prosthesis 100 includes an exterior layer 120 that is relatively firm, a middle pocket 132 that opens to at least one opening 130 and an interior layer 124 that is also relatively soft. Each of the layers, 120 and 124, typically include a silicone gel. The layers are held in flexible chambers that would typically include polyurethane film (typically of about 60 micron to 90 micron thickness) that are sealed together along their periphery along a plurality of welds 126.

In one embodiment, a first polyurethane film 110, a second polyurethane film 112, a third polyurethane film 114 and a fourth polyurethane film 116 are welded together along the weld 126 to form three chambers. The welds 126 have the footprint of a breast form. The material of the exterior layer 120 is placed in a first of the three chambers. The material of the interior layer 124 is placed in a third of the three chambers. A pocket 132 is formed between the second polyurethane film 112 and the third polyurethane film 114, which opens to a plurality of openings 130. A material 134 may be optionally placed in the middle pocket 132 by the user to adjust the shape or the feel of the prosthesis 100. The material 134 could include, for example, a polyester fiberfill.

The material of the exterior layer 120 can include a silicone gel. In one embodiment, the silicone gel includes a two-component addition-cure silicone gel composition that includes a first combination, including a vinyl polymer, a silicone oil and a cross-linker, and a second combination, including a vinyl polymer, a silicone oil and a catalyst (such as a platinum and silicone complex). (One example of suitable silicon gels include prosthesis gels manufactured by Wacker Chemie GmbH.) A typical cross-linker includes a methyl hydrosiloxane polymer.

In one embodiment, gel of the interior layer 124 is uncured (or lightly cured) to facilitate it conforming to the shape of the user's chest when in use. In one embodiment, micro-spheres can be added to one or both of the silicone gels to reduce the weight of the resulting prosthesis 100. Similarly, pigments can be added to give the prosthesis a desired look, such as a natural flesh-tone look. The silicones can also have different degrees of softness.

As shown in FIG. 3, the prosthesis 100 can be formed with several openings 130 to the pocket 132. The embodiment shown includes two side openings 130a and one bottom opening 130b.

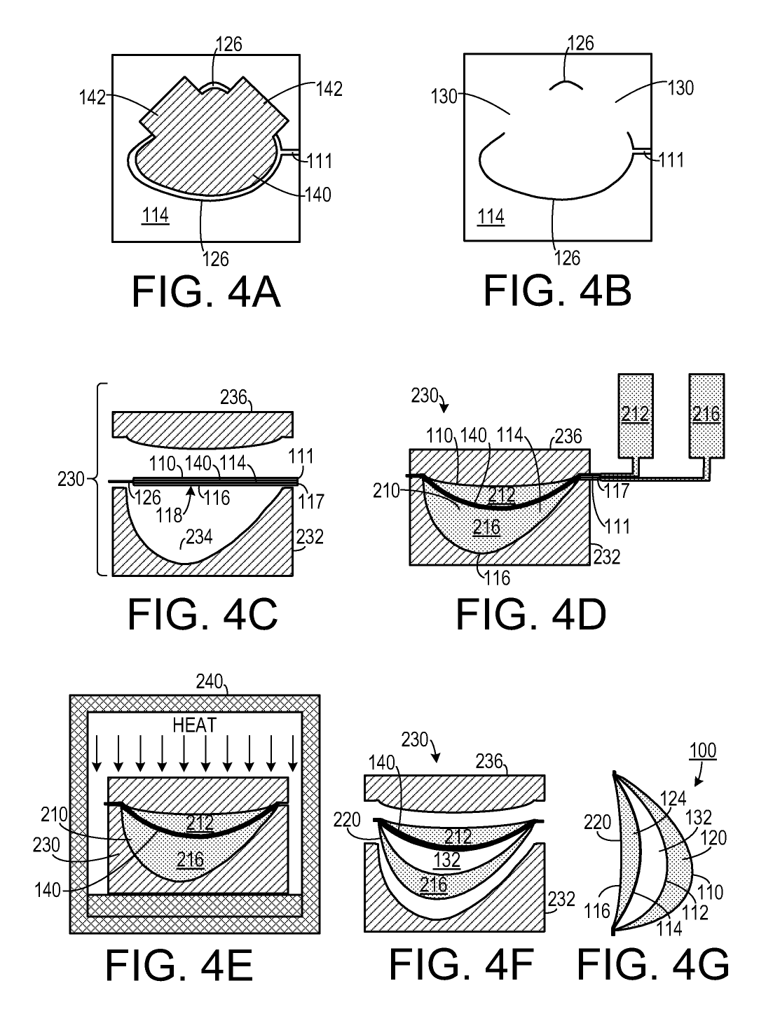

In one embodiment of a method of making a breast form, as shown in FIGS. 4A-4G, four sheets of polyurethane film (110, 112, 114 and 116) are stacked on top of each other, in which a non-stick shape 140 is placed between layer 112 and layer 114. The non-stick shape 140 could, for example, include a polyurethane film to which a release coating has been applied. In other embodiments, the non-stick shape 140 could be made from Polytetrafluoroethylene (PTFE—commercially referred to as “Teflon”) or a PTFE-coated high temperature polymer (such as nylon or polyetherimide, referred to as “Ultem”) or PTFE-coated fiberglass. The non-stick shape 140 includes protrusions 142 that correspond to the desired locations of the openings 130 to the pocket 132.

The four sheets of polyurethane film 110, 112, 114 and 116 are welded together along a weld 126. The weld 126 is made discontinuous between layers 112 and 114 by the non-stick shape 140. This forms an envelope 118 that includes two different chambers with a pocked disposed between the chambers. The envelope 118 has a shape corresponding to a desired breast form. The breast form can be a-symmetric (as in the example shown in FIG. 1) or it can be symmetric. The weld includes a corresponding two sprue inlets (111 and 117) that open the chambers to the outside. The envelope 118 is placed in a mold 230 including a front portion 232 and a back portion 236. The mold 230 defines a cavity 234 having a shape that is complementary to the desired shape of a breast form.

In another embodiment, two sheets of polyurethane film 110 and 112 are stacked on top of each other and are welded together (the weld includes a sprue inlet 111) in a first weld. The non-stick shape 140 is placed on top of the welded two layers 110 and 112 and then two more sheets of polyurethane film 114 and 116 are stacked on top of the non-stick shape 140. A material can be added between film layers 112 and 114 to reduce the chance of the film layers sticking together. Examples of such a material can include talc, a similar fine powder or silicone oil. All four layers are then welded together in a second weld. The second weld essentially traces the first weld. In another embodiment, two sheets of polyurethane film 110 and 112 are welded to form a first sub-envelope and two more sheets of polyurethane film 114 and 116 are welded to form a second sub-envelope. The non-stick shape 140 is placed on top of the first sub-envelope and the second sub-envelope is placed on top of the non-stick shape 140 and the first sub-envelope. The first sub-envelope is then welded to the second sub-envelope along a peripheral weld so as to form a two chamber envelope.

Once in the mold 230, a first silicone gel 212 is injected into the first chamber through the first sprue 111 and a second silicone gel 216 is injected into the second chamber through the second sprue 117. This forms an uncured breast form 210, from which any air is removed. The sprues (111 and 117) are sealed and the mold 230 with the uncured breast form 210 therein is placed in an oven 240 and heated to a sufficient temperature for a sufficient amount of time to cure the first material 212 and the second material 216, thereby forming a cured breast form 220. The exact temperature and time depend on the specific mixture of silicone gel used; however, it can readily be determined from the manufacturer's data sheet. The cured breast form 220 is removed from the oven 240, is allowed to cool, removed from the mold 230, the non-stick shape 140 is removed from the breast form 220 and any excess film is trimmed away from the weld, resulting in a breast prosthesis 100.

The final prosthesis 100 includes a layer 124 closest to the patient that is soft and conforms to the chest of the wearer for comfort. The middle pocket 132 provides a place to add material to adjust the nature of the prosthesis 100. The front layer 120 maintains the exterior shape of the prosthesis 100 so as to give it an appearance of natural breast tissue.

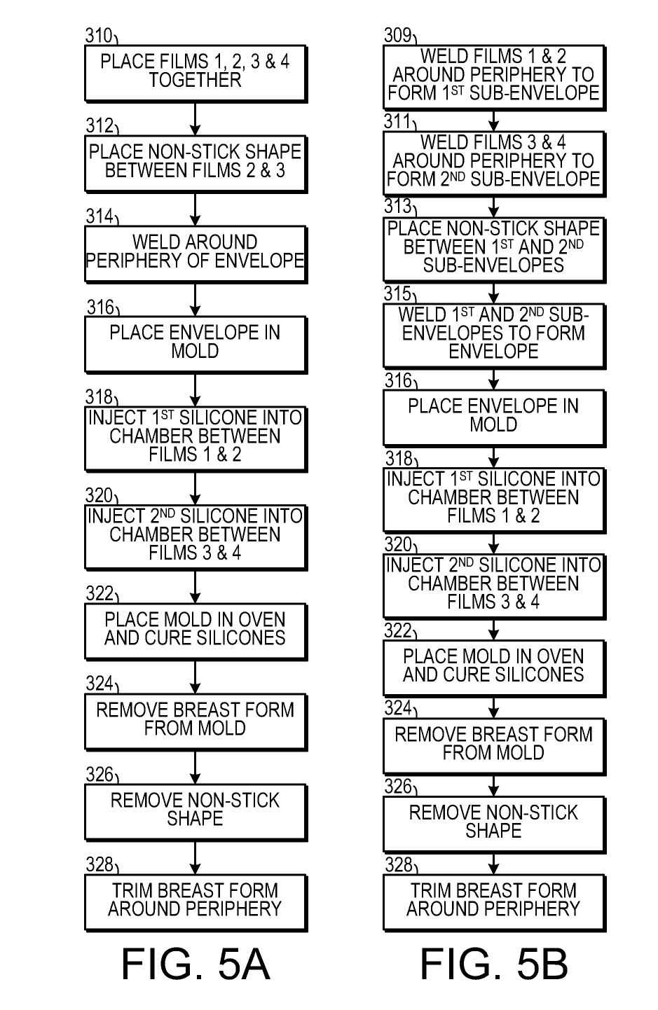

As shown in FIG. 5A, in one method of making a breast prosthesis, four films are placed together 310 with the non-stick shape being placed between the two inner films 312 and the films are welded around a periphery 314 to form an envelope. The envelope is placed in a breast form mold 316, a first silicone is injected into the first chamber 318 and a second silicone is injected into the second chamber 320. The mold is placed into an oven and the silicones are cured 322 as a result of heating. Once cured, the breast form is cooled and is removed from the mold 324. The non-stick shape is removed 326 and is either discarded or recycled. The breast form is then trimmed about the periphery 328. In a slightly different embodiment, as shown in FIG. 5B, two films are welded to form a first sub-envelope 309 and two more films are welded to form a second sub-envelope 311. The non-stick shape is placed between the first sub-envelope and the second sub-envelope 313, and the first sub-envelope is welded to the second sub-envelope 315 along a peripheral weld. Like the previous embodiment, the envelope is placed in a breast form mold 316, a first silicone is injected into the first chamber 318 and a second silicone is injected into the second chamber 320. The mold is placed into an oven and the silicones are cured 322 as a result of heating. Once cured, the breast form is cooled and is removed from the mold 324. The non-stick shape is removed 326 and is either discarded or recycled. The breast form is then trimmed about the periphery 328.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A breast prosthesis for external wearing by a user, comprising:
(a) a first layer that includes two films that are sealed together peripherally so as to define a front chamber;
(b) a second layer that includes two films that are sealed together peripherally so as to define a back chamber, the second layer disposed adjacent to the first layer and defining a pocket therebetween, a portion of a peripheral edge of the second layer sealed to a corresponding portion of a peripheral edge of the first layer so as to define a first opening so that the pocket defined between the first layer and the second layer opens to the first opening, the first opening and the pocket being configured to allow the user to stuff a material into the pocket through the first opening;
(c) a first silicone rubber disposed in the front chamber and cured in an outer shape of a breast; and
(d) a second silicone rubber disposed in the back chamber.

2. The breast prosthesis of claim 1, wherein the two films of the first layer each comprise a polyurethane film.

3. The breast prosthesis of claim 1, wherein the two films of the second layer each comprise a polyurethane film.

4. A method of making a breast prosthesis, comprising the steps of:
(a) placing a first film sheet, a second film sheet, a third film sheet and a fourth film sheet together and placing a non-stick shape between the second film and the third film, the non-stick shape including at least one protrusion extending therefrom;
(b) welding the first film sheet, the second film sheet, the third film sheet and the fourth film sheet along a peripheral weld that is made discontinuous by the at least one protrusion extending from the non-stick shape so as to form an envelope in which the first film sheet and the second film sheet define a first chamber therebetween that opens to a first sprue, and in which the third film sheet and the fourth film sheet define a second chamber therebetween that opens to a second sprue, and in which the second film sheet and the third film sheet define a pocket therebetween that opens to at least one first opening resulting from the weld being made discontinuous by the at least one protrusion;
(c) placing the envelope in a breast form mold having a cavity that is complementary in shape to the breast prosthesis;
(d) injecting a first silicone gel into the first chamber through the first sprue;
(e) injecting a second silicone gel into the second chamber through the second sprue;
(f) curing the first silicone gel so as to form a first silicone rubber and the second silicone gel so as to form a second silicone rubber, thereby forming the breast prosthesis; and
(g) removing the breast prosthesis from the mold; and
(h) removing the non-stick shape from the pocket.

5. The method of claim 4, further comprising the step of placing a filler material in the pocket through the at least one opening into the pocket so as to achieve a desired shape for the breast prosthesis.

6. The method of claim 4, wherein the non-stick shape comprises a film coated with a release coating.

7. The method of claim 4, wherein the first film sheet, the second film sheet, the third film sheet and the fourth film sheet each comprise a polyurethane film.

8. The method of claim 4, wherein the first silicone gel has a softness that is different from the second silicone gel.

9. The method of claim 4, further comprising the step of trimming the envelope around the weld to remove excess film.

10. The method of claim 4, wherein the curing step comprises heating the first silicone gel and the second silicone gel in an oven.

11. A method of making a breast prosthesis, comprising the steps of:
(a) placing a first film sheet and a second film sheet together and welding them along a first weld so as to form a first sub-envelope in which the first film sheet and the second film sheet define a first chamber therebetween that opens to a first sprue;
(b) placing a third film sheet and a fourth film sheet together and welding them along a second weld so as to form a second sub-envelope in which the third film sheet and the fourth film sheet define a second chamber therebetween that opens to a second sprue;
(c) placing the first sub-envelope and the second sub-envelope together so that the first weld is aligned with the second weld and placing a non-stick shape between the first sub-envelope and the second sub-envelope, the non-stick shape including at least one protrusion extending therefrom;
(d) welding the first sub-envelope and the second sub-envelope along a peripheral weld so as to generate an envelope, wherein the peripheral weld is made discontinuous by the at least one protrusion extending from the non-stick shape so as to define a pocket therebetween that opens to at least one first opening resulting from the peripheral weld being made discontinuous by the at least one protrusion;
(e) placing the envelope in a breast form mold having a cavity that is complementary in shape to the breast prosthesis;
(f) injecting a first silicone gel into the first chamber through the first sprue;
(g) injecting a second silicone gel into the second chamber through the second sprue;
(h) curing the first silicone gel so as to form a first silicone rubber and the second silicone gel so as to form a second silicone rubber, thereby forming the breast prosthesis;
(i) removing the breast prosthesis from the mold; and
(j) removing the non-stick shape from the pocket.

12. The method of claim 11, further comprising the step of placing a filler material in the pocket through the at least one opening into the pocket so as to achieve a desired shape for the breast prosthesis.

13. The method of claim 11, wherein the non-stick shape comprises a film coated with a release coating.

14. The method of claim 11, wherein the first film sheet, the second film sheet, the third film sheet and the fourth film sheet each comprise a polyurethane film.

15. The method of claim 11, wherein the first silicone gel has a softness that is different from the second silicone gel.

16. The method of claim 11, further comprising the step of trimming the envelope around the peripheral weld to remove excess film.

17. The method of claim 11, wherein the curing step comprises heating the first silicone gel and the second silicone gel in an oven.
